(19) United States
(12) Patent Application Publication
(10) Pub. No.: US 2022/0081719 A1
(43) Pub. Date: Mar. 17, 2022

(54) METHOD AND APPLICATION THEREOF FOR PREDICTING PROGNOSIS OF CANCER

(71) Applicants: Taipei Veterans General Hospital, Taipei City (TW); NATIONAL YANG-MING UNIVERSITY, Taipei (TW)

(72) Inventors: Jaw-Ching Wu, Taipei City (TW); Yuh-Jin Liang, Taipei City (TW); Yu-Wei Chiou, Taipei City (TW)

(73) Assignees: Taipei Veterans General Hospital, Taipei City (TW); NATIONAL YANG-MING UNIVERSITY, Taipei (TW)

(21) Appl. No.: 17/023,089

(22) Filed: Sep. 16, 2020

(51) Int. Cl. C12Q 1/6886

(52) U.S. Cl. CPC C12Q 1/6886; C12Q 2600/118; C12Q 2600/158

(57) ABSTRACT

The present invention provides a method for predicting a clinical prognosis of a subject having a cancer, especially a liver cancer, by measuring the expression level of at least one biomarker selected from the group consisting of B4GALT6, GLA, GM2A, HEXB and PSAP, and an upregulation of the at least one biomarker is indicative of the subject at increased risk for having a poor clinical prognosis.

### FIELD OF THE INVENTION

The present invention pertains to a method for predicting a clinical prognosis of a subject having a cancer, and its application thereof.

### BACKGROUND OF THE INVENTION

Hepatocellular carcinoma (HCC) is the fifth common and the ranks the third cancer mortality in the world. Unless diagnosed and treated at early stage, the prognosis is poor. Therefore, the identification of novel markers with high sensitivity and specificity for early diagnosis of diseases and predicting prognosis of HCC is urgently needed.

Glycosphingolipids (GSLs) are amphiphilic membrane lipids consisting of a polar oligosaccharide chain attached to a hydrophobic sphingosine-containing ceramide lipid moiety. GSL are essential in many biological recognition processes and mediate cell signal transduction via the organization of lipid rafts. During oncogenesis, altered glycosylation is reflected by the occurrence of tumor-associated carbohydrate antigens on cancer cells. Cancer associated carbohydrates are mostly located on the surface of cancer cells and are therefore potential targets for new diagnostic biomarkers. Therefore, exploration and identification of specific alternations in GSLs patterns should be a promising direction in the cancer biomarker research field, including HCC.

### BRIEF SUMMARY OF THE INVENTION

It is an object of the present invention to provide a method for predicting a clinical prognosis of a subject having a cancer, especially a liver cancer.

The above object is met by the present invention, in which the embodiment of the present invention provides a method for predicting a clinical prognosis of a subject having a cancer, especially a liver cancer, which comprises (i) providing a control cancer-free sample and a test sample from the subject; (ii) measuring the expression level of at least one biomarker selected from the group consisting of B4GALT6, GLA, GM2A, HEXB and PSAP in the control cancer-free sample and in the test sample; (iii) comparing the expression level of at least one biomarker in the test sample to that in the control cancer-free sample and (iv) determining the clinical prognosis of the subject; wherein an elevated expression of the at least one biomarker relative in the test sample to the level of corresponding biomarker in the control cancer-free sample, is indicative of the subject at increased risk for having a poor clinical prognosis.

In one aspect, the present method directs a clinical intervention based on the predicted prognosis. If the subject is identified at increased risk for having the poor clinical prognosis, the method further comprises a step of administering a treatment, which inhibit or reduce the expression level of at least one biomarker selected from the group consisting of B4GALT6, GLA, GM2A, HEXB and PSAP, and/or a step of administering adjuvant therapy.

The present invention also provides a kit for predicting the clinical prognosis of a subject having a cancer, especially a liver cancer, comprising agents for determining the level of at least one biomarker selected from the group consisting of B4GALT6, GLA, GM2A, HEXB and PSAP.

The present invention further provides a method of treating a cancer, especially a liver cancer, in a subject in need thereof, comprising administering a treatment which inhibit or reduce the expression level of at least one biomarker selected from the group consisting of B4GALT6, GLA, GM2A, HEXB and P SAP.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The patent or application file contains at least one color drawing. Copies of this patent or patent application publication with color drawing will be provided by the USPTO upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1A shows total glycan contents of GSLs prepared from the mouse livers are quantified using the resorcinol-HCl staining method.

FIG. 1B shows a thin-layer chromatography (TLC) analysis of total GSLs prepared from mouse livers. Chloroform/methanol/0.2% CaCl2 in water (55:45:10, v/v/v) is used as the developing solvent system. GSLs developed on TLC plates are stained with the resorcinol-HCl reagent.

FIG. 2 shows genes expression profiles of tumor and non-tumor of HBV transgenic mice are analyzed by microarray. Gene B4GALT6 and Gene GLA are significantly up-regulated in mouse liver tumor. Contrary, Gene ST3GAL4 displays down-regulation in mouse liver tumor.

FIG. 3A shows an influence of glycogenes expression on survival. By using publicly available gene expression data sets associated with human HCC, the data mining process is performed. Kaplan-Meier survival plots show that higher expression of glycogenes, including B4GALT6, GLA, GM2A, HEXB and PSAP, results in a worse OS (Overall Survival) in human HCC.

FIG. 3B shows the higher expression of glycogenes ST8SIA5 and ST6GalNAc5 lead to better survival in human HCC.

FIG. 3C shows a representative staining of GM2A in HCC tissue by IHC (100× or 200×). C1 and C2, Recurrence within 2 years, disease free survival 15 months (Score as 3+); C3 and C4, without recurrence within 2 years, disease free interval 225 months (scored as 0).

FIG. 4 shows a receiver operating characteristic (ROC) curves for combined biomarkers in HCC patients. The ROC curve base on the combination of (1) GM2A, PSAP and Twist; (2) PSAP, Snail and Twist; and (3) Snail and Twist are shown. Accuracy is measured by the area under the curve (AUC). The combination of biomarkers with GM2A, PSAP and Twist demonstrate the highest diagnostic accuracy with AUC=0.8825, P<0.0001.

FIG. 5A shows an effect of GM2A overexpression on EMT phenotype. (A) GM2A -overexpressing SNU449 cells showed evidence of EMT, including N-cadherin (Ncad), Fibronectin (FN1) Vimentin, Twist and Snail, upregulation.

FIG. 5B shows an effect of GM2A knockdown on EMT phenotype. GM2A-silencing Mahlavu cells displayed downregulation of Fibronectin (FN1), Twist and Snail, which are all indicators of EMT.

### DETAILED DESCRIPTION OF THE INVENTION

Unless defined otherwise, all technical and scientific terms used herein have the same meaning as commonly understood by a person skilled in the art to which this invention belongs.

As used herein, the indefinite articles “a” and “an” and the definite article “the” are intended to include both the singular and the plural, unless the context in which they are used clearly indicates otherwise.

The present invention is based, at least in part, on the discovery that GSLs related glycogenes, including B4GALT6, GLA, GM2A, HexB, and PSAP, are significantly correlated with the recurrence and overall survival of HCC. Therefore, they can be used to predict the clinical prognosis of HCC and have potential as targets for innovative therapies.

Accordingly, the present invention provides a method for predicting a clinical prognosis of a subject having a cancer, especially a liver cancer.

Particularly, the method comprises the following steps: (i) providing a control cancer-free sample and a test sample from the subject; (ii) measuring the expression level of at least one biomarker selected from the group consisting of B4GALT6, GLA, GM2A, HEXB and PSAP in the control cancer-free sample and in the test sample; (iii) comparing the expression level of at least one biomarker in the test sample to that in the control cancer-free sample and (iv) determining the clinical prognosis of the subject; wherein an elevated expression of the at least one biomarker relative in the test sample to the level of corresponding biomarker in the control cancer-free sample, is indicative of the subject at increased risk for having a poor clinical prognosis.

In one aspect, the present invention provides a method for predicting a clinical prognosis of a subject having a cancer, especially a liver cancer, which comprises (i) providing a control cancer-free sample and a test sample from the subject; (ii) measuring the expression level of at least one biomarker selected from the group consisting of B4GALT6, GLA, GM2A, HEXB and PSAP in the control cancer-free sample and in the test sample; (iii) comparing the expression level of at least one biomarker in the test sample to that in the control cancer-free sample and (iv) determining the clinical prognosis of the subject, wherein an elevated expression of the at least one biomarker relative in the test sample to the level of corresponding biomarker in the control cancer-free sample, is indicative of the subject at increased risk for having a poor clinical prognosis.

In certain embodiments, the present invention provides a method for predicting the likelihood of recurrence of a cancer, especially a liver cancer, which comprises (i) providing a control cancer-free sample and a test sample from the subject; (ii) measuring the expression level of at least one biomarker selected from the group consisting of B4GALT6, GLA, GM2A, HEXB and PSAP in the control cancer-free sample and in the test sample; (iii) comparing the expression level of at least one biomarker in the test sample to that in the control cancer-free sample; and (iv) determining the clinical prognosis of the subject; wherein an elevated expression of the at least one biomarker relative in the test sample to the level of corresponding biomarker in the control cancer-free sample, is indicative of the subject at increased risk for having a recurrence of the cancer.

In certain embodiments, the present invention provides a method for predicting the likelihood of cancer-related death, especially a liver cancer, which comprises (i) providing a control cancer-free sample and a test sample from the subject; (ii) measuring the expression level of at least one biomarker selected from the group consisting of B4GALT6, GLA, GM2A, HEXB and PSAP in the control cancer-free sample and in the test sample; (iii) comparing the expression level of at least one biomarker in the test sample to that in the control cancer-free sample; and (iv) determining the clinical prognosis of the subject; wherein an elevated expression of the at least one biomarker relative in the test sample to the level of corresponding biomarker in the control cancer-free sample, is indicative of the subject at increased risk of cancer-related death.

B4GALT6, GLA, GM2A, HEXB and PSAP as defined herein are herein referred to as “biomarkers” of the invention and are characterized by corresponding SEQ IDs.

In several embodiments, B4GALT6 is also known as Beta-1,4-Galactosyltransferase 6. A preferred B4GALT6 is shown in the amino acid sequences of SEQ ID NO 1 or 2, or the mRNA sequences of SEQ ID NO 3 or 4.

In several embodiments, GLA is also known as Galactosidase Alpha. A preferred GLA is shown in the amino acid sequence of SEQ ID NO 5 or the mRNA sequence of SEQ ID NO 6.

In several embodiments, GM2A is also known as GM2 Ganglioside Activator. A preferred GM2A is shown in the amino acid sequences of SEQ ID NO 7 or 8, or the mRNA sequences of SEQ ID NO 9 or 10.

In several embodiments, HEXB is also known as Hexosaminidase B. A preferred HEXB is shown in the amino acid sequences of SEQ ID NO 11 or 12, or the mRNA sequences of SEQ ID NO 13 or 14.

In several embodiments, PSAP is also known as Sphingolipid Activator Protein-1. A preferred PSAP is shown in the amino acid sequences of SEQ ID NO 15, 16 or 17, or the mRNA sequences of SEQ ID NO 18, 19 or 20.

The term “prognosis” as used herein refers to the prediction of the likelihood of cancer-attributable death or progression, including recurrence, metastatic spread, and drug resistance, of a cancer.

The term “prediction”, “predict” or “predicting” as used herein refers to the likelihood that a subject will have a particular clinical outcome, whether positive or negative. The predictive methods of the present invention can be used clinically to make treatment decisions by choosing the most appropriate treatment modalities for any particular patient. The predictive methods of the present invention are valuable tools in predicting if a patient is likely to respond favorably to a treatment regimen, such as surgical intervention, radiation therapy, or chemical therapy. The prediction may include prognostic factors.

The term “subject” as used herein includes, but is not limited to, human or non-human animals, such as companion animals (e.g. dogs, cats, etc.), farm animals (e.g. cattle, sheep, pigs, horses, etc.), or experimental animals (e.g. rats, mice, guinea pigs, etc.).

In certain aspects, the method involves obtaining a sample from a subject. The method of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from liver tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects the sample is obtained from cystic fluid or fluid derived from a tumor or neoplasm.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein.

The sample may be obtained by methods known in the art. In some embodiments the samples are obtained by biopsy. In other embodiments the sample is obtained by swabbing, scraping, phlebotomy, or any other methods known in the art. In some cases, the sample may be obtained, stored, or transported using components of a kit of the present methods. In some cases, multiple samples, such as multiple liver samples may be obtained for diagnosis by the methods described herein. In other cases, multiple samples, such as one or more samples from one tissue type (for example breast) and one or more samples from another tissue may be obtained for diagnosis by the methods. Samples may be obtained at different times are stored and/or analyzed by different methods. For example, a sample may be obtained and analyzed by routine staining methods or any other cytological analysis methods.

In some embodies, the sample is obtained by an invasive procedure including but not limited to: biopsy, needle aspiration, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

The genes or gene products expression profiles of the present invention consists of a group of genes or gene products, including mRNAs and proteins, that are differentially expressed (e.g., up-regulated or down-regulated) in a subject whose liver cancer is likely to recur after treatment of the primary tumor. Specifically, some of these genes and their encoded proteins are up-regulated (over-expressed) in the subject having liver cancer, whose cancer is likely to recur/metastasize after treatment of the primary tumor, relative to expression of the same genes in normal tissue of the subject or the primary cancer tumors of the subject whose cancer is unlikely to recur/metastasize.

The gene or gene products, including mRNAs and proteins, expression profiles of the present invention thus can be used to predict the likelihood of recurrence of the cancer and/or disease-related death. The present gene or gene products, including mRNAs and proteins, expression profiles also can be used to identify those liver cancer patients requiring adjuvant therapies. mRNA expression levels may be measured through direct isolation or by using a primer or probe relative to the mRNA. Examples of analysis methods for the measuring include reverse transcription polymerase chain reaction (RT-PCR), competitive RT-PCR, real-time RT-PCR, RNase protection assay (RPA), northern blotting, nucleic acid microarray including DNA, and any combination thereof. Compared with a control group, the prognosis of a cancer or the risk of recurrence of a cancer in an individual may be easily determined. Here, the control group may refer to a normal or negative control group including samples of individuals without cancer or completely cured individuals. The control group may also refer to a positive control group including samples of individuals currently suffering from cancer or experiencing recurrence of cancer.

The measurement of the protein analysis may be performed by, for example, western blotting, enzyme linked immunosorbent assay (ELISA), radioimmunoassay (RIA), radioimmunodiffusion, Ouchterlony immunodiffusion, rocket immunoelectrophoresis, tissue immunostaining, immunoprecipitation assay, complement fixation assay, fluorescence-activated cell sorting (FACS), mass spectrometry, magnetic bead-antibody immunoprecipitation, a method using a protein chip, or any combination thereof.

The method of detecting the protein may include comparing the measured protein expression level in the biological sample with that of a normal control group including samples from individuals without cancer or completely cured individuals without any manipulation.

The term “gene” refers to a nucleic acid (e.g., DNA) sequence that comprises coding sequences necessary for the production of a polypeptide, RNA (e.g., including but not limited to, mRNA, tRNA and rRNA) or precursor. The polypeptide, RNA, or precursor can be encoded by a full-length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences”. Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) processed transcript. The mRNA functions during translation to specify the sequence or order of amino acids in a nascent polypeptide.

The term “glycogene” used herein refers to glycosylation-related genes or their gene products, which involve in catalyzing the biosynthesis of different glycoconjugates and saccharide structures, and the transfer of sugar moieties from activated donor molecules to specific acceptor molecules that determines the biosynthesis of glycans.

In some embodiments, the cancer type is a solid cancer type or a hematologic malignant cancer type.

In some embodiments, the cancer type is a metastatic cancer type or a relapsed or refractory cancer type. In some embodiments, the cancer type comprises acute myeloid leukemia (LAML or AML), acute lymphoblastic leukemia (ALL), adrenocortical carcinoma (ACC), bladder urothelial cancer (BLCA), brain stem glioma, brain lower grade glioma (LGG), brain tumor, breast cancer (BRCA), bronchial tumors, Burkitt lymphoma, cancer of unknown primary site, carcinoid tumor, carcinoma of unknown primary site, central nervous system atypical teratoid/rhabdoid tumor, central nervous system embryonal tumors, cervical squamous cell carcinoma, endocervical adenocarcinoma (CESC) cancer, childhood cancers, cholangiocarcinoma (CHOL), chordoma, chronic lymphocytic leukemia, chronic myelogenous leukemia, chronic myeloproliferative disorders, colon (adenocarcinoma) cancer (COAD), colorectal cancer, craniopharyngioma, cutaneous T-cell lymphoma, endocrine pancreas islet cell tumors, endometrial cancer, ependymoblastoma, ependymoma, esophageal cancer (ESCA), esthesioneuroblastoma, Ewing sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal cell tumor, gastrointestinal stromal tumor (GIST), gestational trophoblastic tumor, glioblstoma multiforme glioma GBM), hairy cell leukemia, head and neck cancer (HNSD), heart cancer, Hodgkin lymphoma, hypopharyngeal cancer, intraocular melanoma, islet cell tumors, Kaposi sarcoma, kidney cancer, Langerhans cell histiocytosis, laryngeal cancer, lip cancer, liver cancer, Lymphoid Neoplasm Diffuse Large B-cell Lymphoma [DLBCL), malignant fibrous histiocytoma bone cancer, medulloblastoma, medullo epithelioma, melanoma, Merkel cell carcinoma, Merkel cell skin carcinoma, mesothelioma (MESO), metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, Non-Hodgkin lymphoma, nonmelanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pheochromocytoma and paraganglioma (PCPG), pineal parenchymal tumors of intermediate differentiation, pineoblastoma, pituitary tumor, plasma cell neoplasm/multiple myeloma, pleuropulmonary blastoma, primary central nervous system (CNS) lymphoma, primary hepatocellular liver cancer, prostate cancer such as prostate adenocarcinoma (PRAD), rectal cancer, renal cancer, renal cell (kidney) cancer, renal cell cancer, respiratory tract cancer, retinoblastoma, rhabdomyosarcoma, salivary gland cancer, sarcoma (SARC), Sezary syndrome, skin cutaneous melanoma (SKCM), small cell lung cancer, small intestine cancer, soft tissue sarcoma, squamous cell carcinoma, squamous neck cancer, stomach (gastric) cancer, supratentorial primitive neuroectodermal tumors, T-cell lymphoma, testicular cancer testicular germ cell tumors (TGCT), throat cancer, thymic carcinoma, thymoma (THYM), thyroid cancer (THCA), transitional cell cancer, transitional cell cancer of the renal pelvis and ureter, trophoblastic tumor, ureter cancer, urethral cancer, uterine cancer, uterine cancer, uveal melanoma (UVM), vaginal cancer, vulvar cancer, Waldenstrom macroglobulinemia, or Wilm's tumor. In some embodiments, the cancer type comprises acute lymphoblastic leukemia, acute myeloid leukemia, bladder cancer, breast cancer, brain cancer, cervical cancer, cholangiocarcinoma, colon cancer, colorectal cancer, endometrial cancer, esophageal cancer, gastrointestinal cancer, glioma, glioblastoma, head and neck cancer, kidney cancer, liver cancer, lung cancer, lymphoid neoplasia, melanoma, a myeloid neoplasia, ovarian cancer, pancreatic cancer, pheochromocytoma and paraganglioma, prostate cancer, rectal cancer, squamous cell carcinoma, testicular cancer, stomach cancer, or thyroid cancer.

In one embodiment, the present method directs a clinical intervention based on the predicted prognosis. If the subject is identified at increased risk for having the poor clinical prognosis, the method further comprises a step of administering a treatment, which inhibit or reduce the expression level of at least one biomarker selected from the group consisting of B4GALT6, GLA, GM2A, HEXB and PSAP, and/or a step of administering adjuvant therapy.

The term “adjuvant therapy” as used herein refers to the treatment that is given in addition to the primary, main or initial treatment. For example, adjuvant therapy is an additional treatment usually given after surgery where all detectable disease has been removed, but where there remains a statistical risk of relapse due to occult disease.

In one embodiment, the present invention provides a kit for carrying out any of the method disclosed herein for determining a clinical prognosis of a subject having a cancer, especially a liver cancer.

In some embodiments, the kit comprises one or more reagents for determining the expression level of at least one biomarker selected from the group consisting of B4GALT6, GLA, GM2A, HEXB and PSAP in a sample.

In some embodiments, the present invention provides a kit for predicting the clinical prognosis of a subject having a cancer, comprising agents for determining the level of at least one biomarker selected from the group consisting of B4GALT6, GLA, GM2A, HEXB and PSAP.

In some embodiments, kits can be used to evaluate one or more nucleic acid and/or polypeptide molecules. In some embodiments, there are kits for evaluating gene expression, protein expression, or protein activity in a sample.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, 20×, 50×, 100× or more.

Kits for using probes, polypeptide detecting agents, and/or inhibitors or antagonists of the disclosure for prognostic or diagnostic applications are included. Specifically, contemplated are any such molecules corresponding to any nucleic acid or polypeptide identified herein.

In certain aspects, negative and/or positive control agents are included in some kit embodiments.

In further aspects, kits may be used for analysis of a sample by assessing a nucleic acid or polypeptide profile for a sample comprising, in suitable container means, two or more RNA probes, or a polypeptide detecting agent, wherein the RNA probes or polypeptide detecting agent detects nucleic acids or polypeptides described herein. Furthermore, the probes, detecting agents and/or inhibiting reagents may be labeled. Labels are known in the art and also described herein. In some embodiments, the kit can further comprise reagents for labeling probes, nucleic acids, and/or detecting agents. The kit may also include labeling reagents, including at least one of amine-modified nucleotide, poly(A) polymerase, and poly(A) polymerase buffer. Labeling reagents can include an amine-reactive dye. Certain aspects also encompass kits for performing the diagnostic or therapeutic methods. Such kits can be prepared from readily available materials and reagents. For example, such kits can comprise any one or more of the following materials: enzymes, reaction tubes, buffers, detergent, primers, probes, antibodies. In a particular embodiment, these kits allow a practitioner to obtain samples by the methods disclosed herein. In another particular embodiment, these kits include the needed apparatus for performing RNA extraction, RT-PCR, and gel electrophoresis.

In a particular aspect, these kits may comprise a plurality of agents for assessing the differential expression of a plurality of biomarkers, wherein the kit is housed in a container. The agents in the kit for measuring biomarker expression may comprise a plurality of PCR probes and/or primers for qRT-PCR and/or a plurality of antibody or fragments thereof for assessing expression of the biomarkers. In another embodiment, the agents in the kit for measuring biomarker expression may comprise an array of polynucleotides complementary to the mRNAs of the biomarkers. Possible means for converting the expression data into expression values and for analyzing the expression values to generate scores that predict clinical prognosis may be also included.

In one embodiment, the present invention provides a method of treating cancer in a subject in need thereof, comprising administering a treatment which inhibit or reduce the expression level of at least one biomarker selected from the group consisting of B4GALT6, GLA, GM2A, HEXB and PSAP.

In another embodiment, provided herein is a method of prediction cancer progression in a subject in order to determine an optimal treatment, the method comprising the step of obtaining a sample from the subject and measuring the expression profile of at least one biomarker selected from the group consisting of B4GALT6, GLA, GM2A, HEXB and PSAP, in the sample, wherein measuring a biomarker expression level in the subject over the levels observed in that of a control sample enables the predicting the progress of the cancer in the subject, and wherein a composition or mixture of compositions of the present invention is administered at predetermined time that will maximize therapeutic efficacy.

In another embodiment, the treatment decisions could be made by choosing the most appropriate treatment modalities for any particular subject.

In one embodiment, the method of treating cancer disclosed herein comprises administering a treatment, which is a small molecule inhibitor, a polypeptide inhibitor, an antagonistic antibody, or a nucleic acid inhibitor, capable of decreasing or inhibiting the expression of biomarker, to a subject in need thereof. Preferably, the treatment targets at least one biomarker selected from the group consisting of B4GALT6, GLA, GM2A, HEXB and PSAP.

The present invention is further illustrated by the following examples, which are provided for the purpose of demonstration rather than limitation.

### EXAMPLES

Materials and Methods

1. GSL Extraction, HPTLC Analysis

GSLs were extracted as described as our previously publications⁽¹⁻⁴⁾. In brief, 2×10⁸ cells or 0.2-0.5 mg tissue were extracted by successive sonication in the following four solvents (each 10 mL): (i) chloroform/methanol (CM) (1:1), (ii) isopropanol/hexane/water (IHW) (55:25:20, lower phase), (iii) IHW (55:25:20, lower phase), and (iv) CM (1:1). The combined extracts were evaporated and dissolved in 6 mL CM (2:1). The solution was added with 1 mL water to give CM/water (CMW) 4:2:1, shaken, and allowed to separate into upper and lower phases. The lower phase was added with 3 mL CM/0.1% NaCl (1:10:10), shaken, and allowed to separate into upper and lower phases. This step, known as the Folch partition, were repeated three times. The upper phases were combined, washed with 0.5 mL CM (2:1), evaporated, and solubilized in distilled water, and the resulting solution were applied to a Sep-Pak C18 cartridge (Varian) for desalting. GSLs were analyzed using HPTLC plates (EMD Bioscience) and developed in a solvent system of CM/0.5% aqueous CaCl₂) (50:40:10). GSLs were visualized by spraying with 0.5% orcinol in 1 M sulfuric acid.

2. Mass Spectrometric Analysis of GSLs

MALDI-TOF MS profiling analyses of permethylated GSLs were performed on a TOF/TOF 5800 system (Sciex; Canada) using 2,5-dihydroxybenzoic acid as matrix (10 mg/mL in 50% acetonitrile). Permethylated derivatives were dissolved in 50% acetonitrile solution. An aliquot of each sample solution was premixed with an equal amount of matrix solution, and spotted on a MALDI plate.

Each MALDI-TOF MS spectrum were acquired automatically in 2000 laser shots with random sampling acquisition.

3. Gene Expression Array and Data Analysis

To identify candidate genes associated with recurrent HCC, microarray analysis was performed using Human Genome U133 Plus 2.0 arrays (Affymetrix) as per the manufacturer's protocol. Total RNA sample preparation, cRNA probe preparation, and array hybridization were performed as described previously⁽⁵⁾. Data analysis is described in the preceding section. Raw data of CEL files were preprocessed using the R statistical programming language (www.r-project.org), and normalized gene expression values were obtained using the RMA algorithm of Bioconductor affy package⁽⁶⁾. Genes differentially expressed in contrast groups were identified using Bioconductor limma package⁽⁷⁾. A false discovery rate algorithm⁽⁸⁾ was applied to calculate corresponding adjusted p-values. Probe sets with adjusted p-values ≤0.01 were identified as primary candidate genes from comparisons of contrast groups.

4. Quantitative Reverse Transcription PCR (qRT-PCR)

Total RNA was extracted using a RNeasy Plus Mini Kit (Qiagen). The first strand of cDNA was prepared from 5 μg RNA using SuperScript III first-strand Synthesis SuperMix (Invitrogen) with random primers, according to the manufacturer's instructions. Real-time qRT-PCR was performed using 200 ng cDNA in a thermal cycler (ABI PRISM 7900 Sequence Detection System; Applied Biosystems) according to the manufacturer's protocol. Relative quantities of mRNAs were determined using the comparative threshold number (AACt method), with genes for (3-actin, GAPDH, and Ups11 as reference genes.

5. Glycogene-Overexpressing and Glycogene-Knockdown Cell Lines

Glycogenes human cDNA ORF clones were from OriGene. A full-length cDNA fragment was PCR-amplified and sub-cloned into a lentiviral vector pLAS2w.Pbsd (National RNAi Core Facility; Taipei, Taiwan) or mammalian expression vector pCMV-Tag2b (Stratagene). A shGlycogenes clones (small hairpin targeted different glycogenes; pLKO.1 vector) was from National RNAi Core Facility. Lentivirus production was performed in a HEK293T cell viral package system. Cell lines Huh7, Malavue and SNU449 were transduced with glycogene full-length cDNA or glycogene-short hairpin (sh) sequence containing lentivirus with multiplicity of infection (MOI)=2. Stable clones were selected with blasticidin (5 μg/mL) for pLAS2w.Pbsd vector, puromycin (1 μg/mL) for pLKO.1 vector, and G418 (500 μg/mL) for pCMV-Tag 2B vector. Antibiotic-resistant clones were pooled to avoid clonal variation.

6. Immunohistochemistry (IHC) Staining

IHC staining was performed on paraffin-embedded clinical tissue samples obtained from the Pathology Dept. of TVGH. Sections from paraffin-embedded tissue blocks were processed and analyzed as described previously^{(5,9)}. In brief, sections were antigen-retrieved, endogenous peroxidase was inactivated, sections were incubated with anti-glycogenes primary antibody and processed by Super Sensitive IHC detection system (Biogenex; Fremont, Calif., USA), signals were detected based on 3-amino-9-ethylcarbazole (AEC) subtraction, and sections were counterstained with hematoxylin. IHC staining results were scored independently by two experienced specialists who were blinded to the clinical data.

7. Kaplan-Meier Survival Analysis

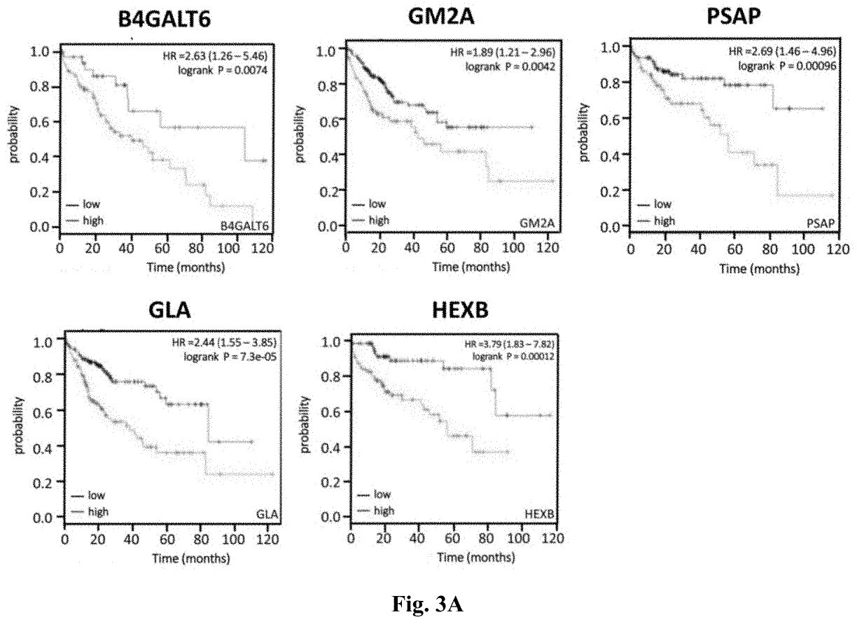

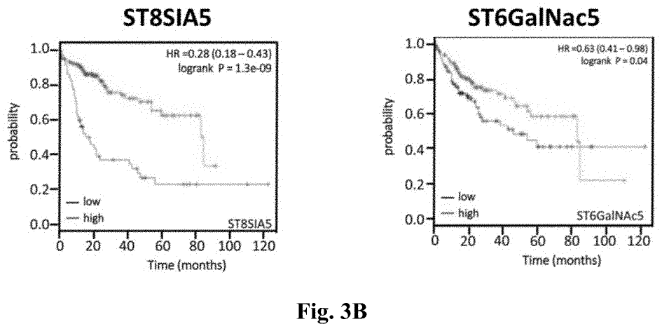

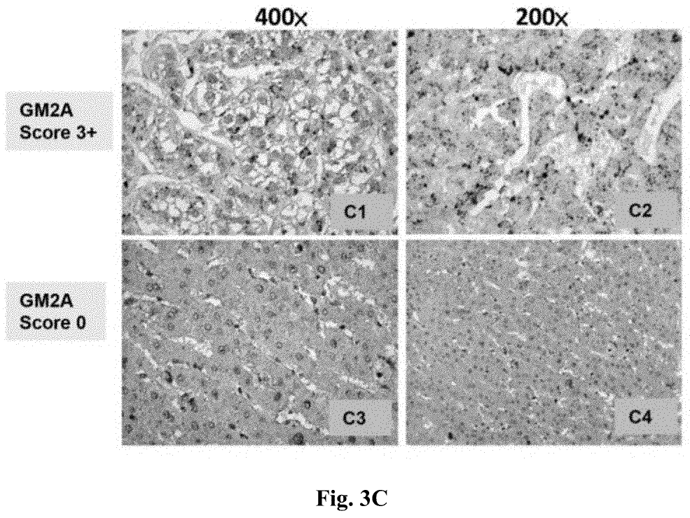

The Kaplan-Meier survival analysis was performed by the KM plotter analysis tool⁽¹⁰⁾ for the OS (Overall Survival) rate of patients with HCC. The KM plotter database has combined the published miRNA expression, OS and clinical data from The Cancer Genome Atlas (http://cancergenome.nih.gov), Gene Expression Omnibus (http://www.ncbi.nlm.nih.gov/geo/), European Genome-Phenome Archive (https://www.ebi.ac.uk/ega/home) and PubMed (http://www.pubmed.com). The statistical outcomes calculated from the database, including hazard ratio (HR), 95% confidence intervals and log rank P-values, were also included in the FIG. 3.

P<0.05 was considered to indicate a statistically significant difference.

8. Receiver Operating Characteristic (ROC) Curves

The ROC analysis was performed in the R statistical environment (http://www.r-project.org) using the ROC Bioconductor library (http://www.bioconductor.org). A Bonferroni correction was applied to account for multiple testing. The statistical significance was set at p<0.001.

9. Statistical Analysis

Data were analyzed by one-way ANOVA followed by Newman-Keuls multiple comparison post hoc test to compare all groups with control group, or by unpaired Student's t-test to compare designated pairs of groups, using Prism 5 software program (GraphPad). Differences were considered significant at p<0.05.

### Example 1 Glycosphingolipids (GSLs) Expression in the Liver Tissue of Hybrid HBV Transgenic Mice

This study tested the possibility that alterations in glycosphingolipid (GSL) patterns correlated with HBV related hepatocarcinogenesis by analyzing GSL changes in the liver tissue of hybrid of HBV transgenic mice with expression of HBV genome and one allele knocked out of miR-122 gene. Purified GSLs from liver tissues of non-transgenic or transgenic mice are analyzed by high performance thin layer chromatography (HPTLC) and matrix-assisted laser desorption/ionization mass spectrometry (MALDI-MS). In addition, the differentially expressed genes which directly involved in GSLs assembly between transgenic and non-transgenic mice are analyzed by microarray methods.

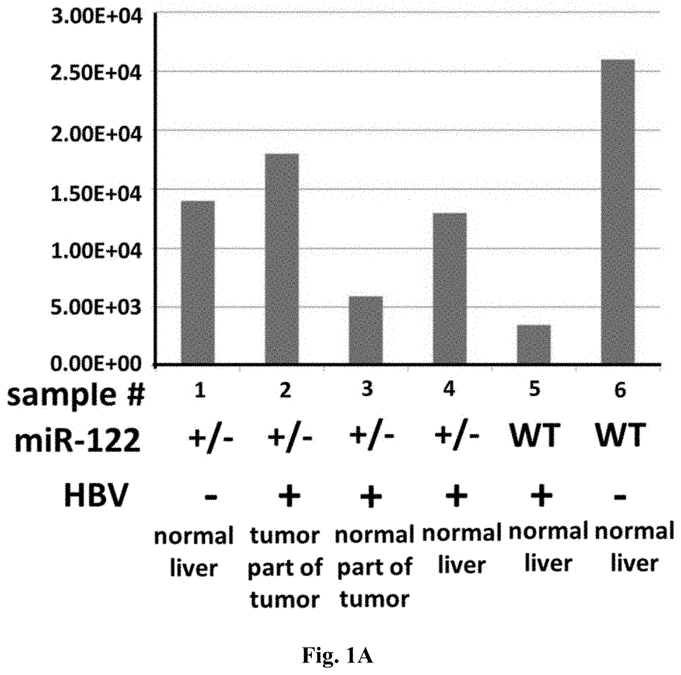

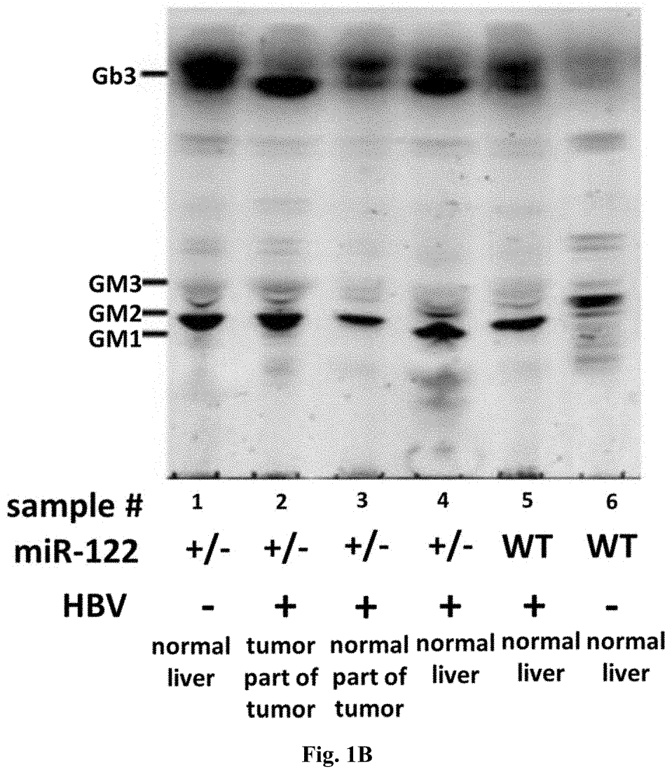

The results showed that he GSLs content drastically decreased in the transgenic mice (FIG. 1A, sample #1-5), compared with non-transgenic mouse (FIG. 1A, sample #6). The GSLs expression patterns switched from heterogeneous, consisting of multiple slowly migrating bands to the more homogeneous, containing more abundant fast migrating bands on TLC during tumor progression in the hybrid HBV transgenic mice (FIG. 1B). MALDI-MS results from the Folch partition upper phase identified GA1, GM2, GD3, GM1, GD2 and GD1 are expressed in the non-transgenic mice. However, GM2 is predominantly expressed in the transgenic mice.

### Example 2 Changes in Gene Expression of Glycogenes in the Liver Tissue of Hybrid of HBV Transgenic Mice

By using microarray analysis, we observed that the mRNA level for glycosyltransferases, B4GALT6 and A4GALT, which are responsible for Gb3 synthesis, were up-regulated in transgenic mice. The results explained that Gb3 is more abundant in the liver tissue of transgenic mice than non-transgenic mice. The mRNA level for many key GSL glycosidases and their co-factors are significantly up-regulated. Several glycosyltransferases which responsible for chain elongation of GSLs synthesis, are down-regulated in transgenic mice. The results explained that the increased of short glycan chain GSLs in the liver of transgenic mice than non-transgenic mice.

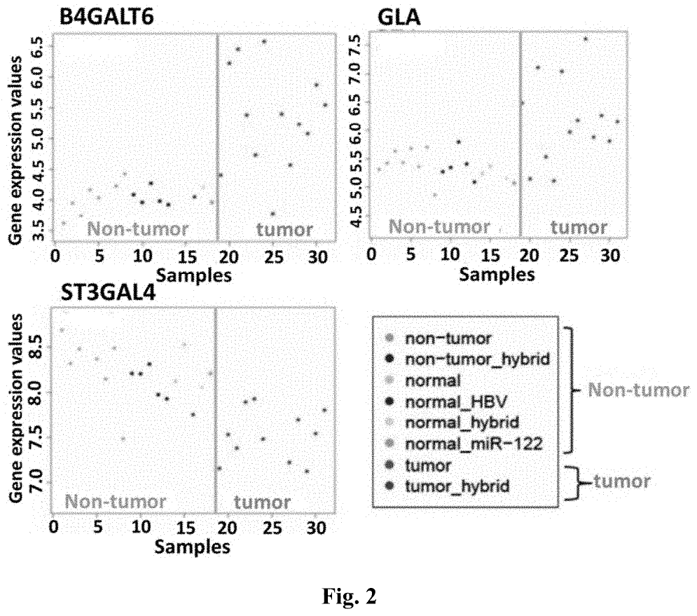

By grouping the mouse livers microarray data into two categories of tumor and non-tumor, we found that glycogenes (genes that are directly involved in glycan assembly) B4GALT6 and GLA are significantly up-regulated, while glycogene ST3GAL4 is down-regulated in the liver tumor (FIG. 2).

### Example 3 Influence of Glycogenes Expression on Survival

A data mining process is performed by using publicly available gene expression data sets associated with human HCC. Kaplan-Meier survival analysis show that the glycogenes, including B4GALT6, GLA, GM2A, HEXB and PSAP, up-regulated in mouse liver results in a worse overall survival, while the higher expressions of ST8SIA5 and ST6GalNAc5 lead to better survival in human HCC (FIG. 3). The results demonstrated that the glycogenes which changed in the HBV-related transgenic mouse model are well correlated with HCC patients' survival time (FIG. 3).

Using candidate gene-specific antibodies, immunohistochemical (IHC) staining was performed on HCC samples of our own cohort to further verify whether the candidate genes were associated with liver cancer. High GM2A expression significantly correlated with tumor recurrence and shorter OS (FIG. 3C).

### Example 4 Receiver Operating Characteristic (ROC) Curves for Combined Biomarkers in HCC Patients

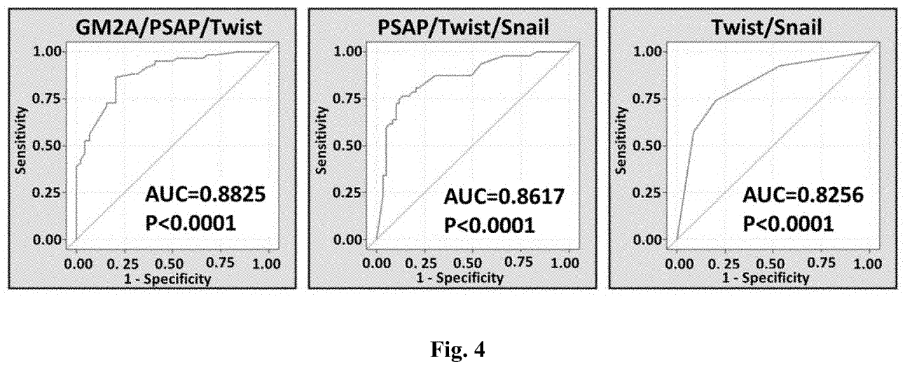

The ROC curve base on the combination of (i) GM2A, PSAP and Twist (ii) PSAP, Snail and Twist (iii) Snail and Twist are shown in FIG. 4. Accuracy is measured by the area under the curve (AUC). The combination of biomarkers with GM2A, PSAP and Twist demonstrate the highest diagnostic accuracy with AUC=0.8825, P<0.0001.

### Example 5 Effects of GM2A Overexpression or Knockdown on EMT Phenotype

The 3D tumorsphere formation method were used to enrich the cancer stem cell phenotypes and the mRNA level of EMT markers and different glycogenes were measured by Q-RT-PCR. We found that GM2A upregulate during sphere formation and link to the EMT phenotype in Mahlavu cells.

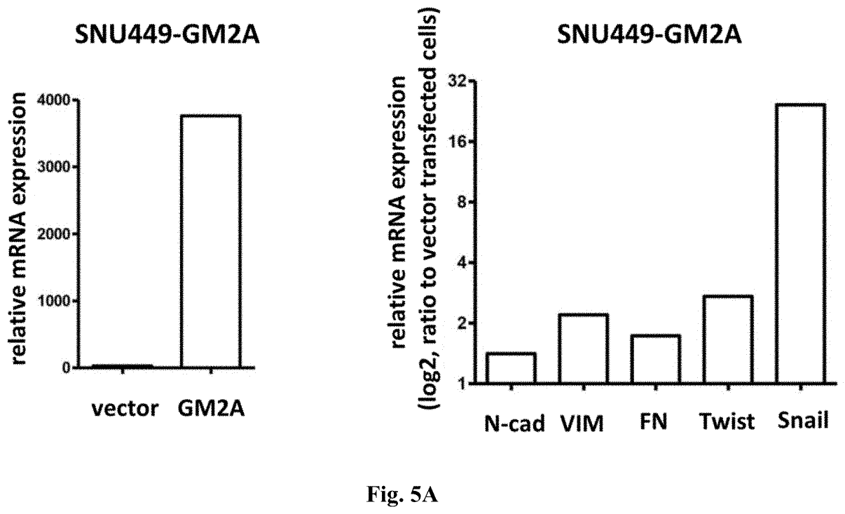

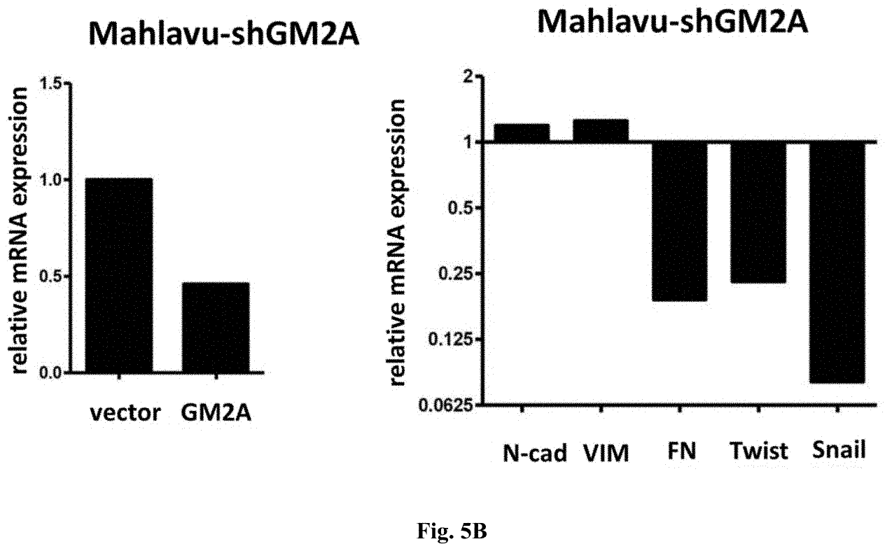

GM2A-overexpressing SNU449 cells showed evidence of EMT, including N-cadherin (Ncad), Fibronectin (FNJ) Vimentin, Twist and Snail, upregulation (FIG. 5A). In contrary, GM2A-silencing Mahlavu cells displayed downregulation of Fibronectin (FNJ), Twist and Snail, which are all indicators of EMT (FIG. 5B).

Taken together, we proposed that the dramatic changes of GSL pattern and the responsible glycogenes expression in the HBV-related transgenic mouse model could be used as a potential predictor of survival on human liver cancer. The results demonstrated that HBV genome expression in HBV transgenic mice dramatically changes of GSL pattern and their responsible glycogenes expression, and the expression of specific glycogene was associated with EMT phenotype and clinical outcomes. Therefore, GSLs related biomarkers, including B4GALT6, GLA, GM2A, HEXB and PSAP could be used for the prediction of HCC development and progression.

The above description merely relates to preferred embodiments in the present invention, and it should be pointed out that, for a person of ordinary skill in the art, some improvements and modifications can also be made under the premise of not departing from the principle of the present invention, and these improvements and modifications should also be considered to be within the scope of protection of the present invention.

### REFERENCES

1. Liang Y J, Wang C Y, Wang I A, et al. Interaction of glycosphingolipids GD3 and GD2 with growth factor receptors maintains breast cancer stem cell phenotype. Oncotarget 2017; 8:47454-47473.
2. Liang Y J, Ding Y, Levery S B, et al. Differential expression profiles of glycosphingolipids in human breast cancer stem cells vs. cancer non-stem cells. Proc Natl Acad Sci USA 2013; 110:4968-73.
3. Liang Y J, Kuo H H, Lin C H, et al. Switching of the core structures of glycosphingolipids from globoand lacto- to ganglio-series upon human embryonic stem cell differentiation. Proc Natl Acad Sci USA 2010; 107:22564-9.
4. Liang Y J, Yang B C, Chen J M, et al. Changes in glycosphingolipid composition during differentiation of human embryonic stem cells to ectodermal or endodermal lineages. Stem Cells 2011; 29:1995-2004.
5. Wang H W, Hsieh T H, Huang S Y, et al. Forfeited hepatogenesis program and increased embryonic stem cell traits in young hepatocellular carcinoma (HCC) comparing to elderly HCC. BMC Genomics 2013; 14:736.
6. Gautier L, Cope L, Bolstad B M, et al. affy—analysis of Affymetrix GeneChip data at the probe level. Bioinformatics 2004; 20:307-15.
7. Smyth G K. limma: Linear Models for Microarray Data. In: Gentleman R, Carey V, Huber W, Irizarry R, Dudoit S, eds. Bioinformatics and Computational Biology Solutions Using R and Bioconductor: Springer New York, 2005:397-420.
8. Benjamini Y, Hochberg Y. Controlling the False Discovery Rate: A Practical and Powerful Approach to Multiple Testing. Journal of the Royal Statistical Society. Series B (Methodological) 1995; 57:289-300.
9. Yang M H, Chen C L, Chau G Y, et al. Comprehensive analysis of the independent effect of twist and snail in promoting metastasis of hepatocellular carcinoma. Hepatology 2009; 50:1464-74.
10. Gyorffy B, Surowiak P, Budczies J, et al. Online survival analysis software to assess the prognostic value of biomarkers using transcriptomic data in non-small-cell lung cancer. PLoS One 2013; 8:e82241.

What is claimed is:

1. A method for predicting a clinical prognosis of a subject having a cancer, comprising
providing a control cancer-free sample and a test sample from the subject;
measuring the expression level of at least one biomarker selected from the group consisting of B4GALT6, GLA, GM2A, HEXB and PSAP in the control cancer-free sample and in the test sample;
comparing the expression level of at least one biomarker in the test sample to that in the control cancer-free sample; and
determining the clinical prognosis of the subject; wherein an elevated expression of the at least one biomarker relative in the test sample to the level of corresponding biomarker in the control cancer-free sample, is indicative of the subject at increased risk for having a poor clinical prognosis.

2. The method of claim 1, wherein the cancer is a solid cancer type or a hematologic malignant cancer.

3. The method of claim 1, wherein the cancer comprises gastrointestinal cancer.

4. The method of claim 3, wherein the cancer is liver cancer.

5. The method of claim 1, wherein the expression level of at least one biomarker comprises mRNA or protein expression level.

6. The method of claim 1, further comprising a step of administering a treatment, which inhibit or reduce the expression level of at least one biomarker selected from the group consisting of B4GALT6, GLA, GM2A, HEXB and PSAP, if the subject is identified at increased risk for having the poor clinical prognosis.

7. The method of claim 1, further comprising a step of administering adjuvant therapy if the subject is identified at increased risk for having the poor clinical prognosis.

8. A kit for predicting the clinical prognosis of a subject having a cancer, comprising agents for determining the level of at least one biomarker selected from the group consisting of B4GALT6, GLA, GM2A, HEXB and PSAP.

9. A method of treating cancer in a subject in need thereof, comprising administering a treatment which inhibit or reduce the expression level of at least one biomarker selected from the group consisting of B4GALT6, GLA, GM2A, HEXB and PSAP.

10. The method of claim 9, wherein the treatment comprises a small molecule inhibitor, a polypeptide inhibitor, an antagonistic antibody, or a nucleic acid inhibitor, capable of decreasing or inhibiting the expression of at least one biomarker selected from the group consisting of B4GALT6, GLA, GM2A, HEXB and PSAP.
